(19) United States
(12) Patent Application Publication
(10) Pub. No.: US 2014/0266695 A1
(43) Pub. Date: Sep. 18, 2014

(54) SYSTEM AND METHOD FOR DISINFECTING AND CHARGING A SENSOR

(71) Applicant: COVIDIEN LP, Mansfield, MA (US)

(72) Inventors: Paul S. Addison, Edinburgh (GB); James N. Watson, Dunfermline (GB); James H. Dripps, West Linton (GB); George K. Manning, Linlithgow (GB)

(73) Assignee: COVIDIEN LP, Mansfield, MA (US)

(21) Appl. No.: 13/837,627

(22) Filed: Mar. 15, 2013

(51) Int. Cl. A61B 5/00; A61B 5/1455

(52) U.S. Cl. CPC A61B 5/7405; A61B 5/14551
USPC 340/539.12; 600/323

(57) ABSTRACT

Methods and systems are provided for disinfecting and recharging a sensor. The sensor may be a wireless sensor, or the sensor may be configured to operate in a wireless mode and in a wired mode. The system may include a charging device configured to provide power to the sensor and/or to recharge a power source of the sensor. The sensor may include a proximity detector configured to provide information relating to a proximity of the sensor to the charging device. Additionally, the sensor may include additional sensors configured to provide information relating to a disinfection process utilized to disinfect the sensor.

### BACKGROUND

The present disclosure relates generally to medical devices, and more particularly, to medical devices that monitor physiological parameters of a patient, such as pulse oximeters.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters and other types of monitoring devices may use either disposable sensors, which are discarded after a single use, or reusable sensors. Reusable sensors may lower the overall cost of the sensor per use; however, reusable sensors must be thoroughly disinfected before use with a new patient and/or periodically throughout use with the same patient. Unfortunately, users may experience difficulty in remembering whether a sensor has been disinfected (e.g., before providing the sensor to a new patient) and/or how much time has elapsed since a sensor was last disinfected.

### BRIEF DESCRIPTION OF THE DRAWINGS

Advantages of the disclosed techniques may become apparent upon reading the following detailed description and upon reference to the drawings in which:

FIG. 1 illustrates a perspective view of a patient monitoring system including a patient monitor and a sensor, in accordance with an embodiment;

FIG. 2 illustrates a block diagram of the patient monitoring system of FIG. 1, including the patient monitor and the sensor of FIG. 1, and an optical scanner, in accordance with an embodiment;

FIG. 3 illustrates a flow diagram of a method for determining a disinfection condition of the sensor of FIG. 1, in accordance with an embodiment;

FIG. 4 illustrates a schematic diagram of a disinfecting system for disinfecting the sensor of FIG. 1, in accordance with an embodiment;

FIG. 5 illustrates a flow diagram of a method for determining information related to a disinfection process for the sensor of FIG. 1, in accordance with an embodiment;

FIG. 6 illustrates a block diagram of patient monitoring system of FIG. 1, including the patient monitor and the sensor of FIG. 1, and a charging device, in accordance with an embodiment; and

FIG. 7 illustrates a flow diagram of a method for providing an indication to couple the sensor of FIG. 1 to a charging device, in accordance with an embodiment.

### DETAILED DESCRIPTION OF THE SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, reusable sensors may be disinfected periodically. For example, the sensor may be disinfected periodically throughout use with the same patient and/or before use with a new patient. However, users may experience difficulty in remembering whether a sensor has been disinfected (e.g., before providing the sensor to a new patient) and/or how much time has elapsed since a sensor was last disinfected. Additionally, while reusable sensors may be periodically disinfected, a user may not be provided with information regarding the disinfection process for the sensor. Information regarding the disinfection process may be useful for quality control purposes. For example, if a disinfection system is faulty, it may be desirable to disinfect the sensor again and/or provide the sensor to a different disinfection system.

To address these issues, the present embodiments describe sensors that may be equipped with processing circuitry configured to monitor and/or determine a disinfection condition and to provide an indication of the disinfection condition. In particular, a sensor may include at least two disinfection conditions (e.g., states or statuses). A first disinfection condition may indicate that continued sensor operation may be acceptable, and a second disinfection condition may indicate that disinfecting or disposing of the sensor may be desirable. In some embodiments, a reusable sensor may be configured to determine the time that has elapsed since the last disinfection. The reusable sensor may provide an indication that disinfection may be desirable if the time that has elapsed since the last disinfection is greater than a predetermined threshold. Similarly, a disposable sensor (e.g., single use sensor) may be configured to determine the time that has elapsed since the sensor has been in use and, further, may be configured to provide an indication that disposal of the sensor may be desirable if the time that has elapsed is greater than a predetermined threshold. Thus, the present embodiments may alert the user when it may be desirable to disinfect a reusable sensor and/or to dispose of a disposable sensor.

Additionally, the sensor may include one or more additional sensors that may be configured to provide information (e.g., quality information) regarding a disinfection process utilized to disinfect the sensor. For example, in certain embodiments, the sensor may include a temperature sensor and/or a pressure sensor, which may detect that the sensor is being disinfected (e.g., via high temperatures and/or pressures). Additionally, the sensor may be equipped with a transceiver to wirelessly communicate with a disinfection system. In this manner, the sensor may determine (e.g., via a signal from the disinfection system) that it is being disinfected. Further, the sensor may be configured to provide an indication of a completed disinfection process to the user. Additionally, the sensor may be configured to provide an indication relating to the quality of the completed disinfection process to the user. Thus, the present embodiments may provide the user with a confirmation of a completed disinfection process and information regarding the disinfection process. Embodiments such as these are discussed with respect to FIGS. 1-5.

However, monitoring the disinfection condition of the sensor and monitoring the disinfection process may increase the power consumption of the sensor. Additionally, the sensor may operate using battery power during certain disinfection processes that may not be conducive to cables. As a result, for embodiments in which the sensor utilizes a battery as a power source, problems may exist in which the battery may drain, and the battery must be recharged or replaced to continue sensor operation. Accordingly, it may be desirable to provide sensors which may operate using both battery power and wired power from a charging device. For example, the sensor may include a power connector to receive a cable of a charging device and may operate using power received from the charging device while the cable is coupled to the power connector. The sensor may operate using battery power while the cable is not connected to the power connector. To minimize battery drainage, it may be desirable to minimize battery use and operate using wired power when the reusable sensor is within range of a charging device. Occasionally, the user and/or the patient may forget to attach the sensor to the cable of the charging device when the sensor is within range of the charging device.

To address this issue, the present embodiments describe a sensor that detects when it is within a predetermined range of a charging device and provides an indication to a user that the sensor is within the predetermined range. For example, the charging device may include a short-range wireless transmitter, and the sensor may include a short-range wireless reader that may read a signal transmitted by the short-range wireless transmitter when the sensor is within a predetermined range of the charging device. Additionally, the sensor may be configured to provide an indication to the user based in part upon the received signal from the charging device. For example, the sensor may include an indicator light that may emit and/or flash light in response to determining that the sensor is within the predetermined range. Embodiments such as these are discussed with respect to FIGS. 6 and 7.

With the foregoing in mind, FIG. 1 illustrates an embodiment of a patient monitoring system 10 that may include a patient monitor 12 and a sensor 14, such as a pulse oximetry sensor, to monitor physiological parameters of a patient. By way of example, the sensor 14 may represent a MAXFAST™, NEOMAX™, or other pulse oximetry sensor available from Covidien LP. Although the depicted embodiments relate to sensors for use on a patient's fingertip, toe, or earlobe, it should be understood that, in certain embodiments, the features of the sensor 14 as provided herein may be incorporated into sensors for use on other tissue locations, such as the forehead and/or temple, the heel, stomach, chest, back, or any other appropriate measurement site. In addition, although the embodiment of the patient monitoring system 10 illustrated in FIG. 1 relates to photoplethysmography or pulse oximetry, the patient monitoring system 10 may be configured to obtain a variety of medical measurements with a suitable medical sensor. For example, the patient monitoring system 10 may additionally or alternatively be configured to perform regional oximetry, determine patient electroencephalography (e.g., a bispectral (BIS) index), or any other physiological parameter such as tissue water fraction or hematocrit.

The sensor 14 may include one or more emitters 16 and one or more detectors 18 to acquire a physiological signal corresponding to one or more physiological parameters of a patient. For pulse oximetry applications, the emitter 16 may transmit at least two wavelengths of light (e.g., red and/or infrared (IR)) into a tissue of the patient. The detector 18 may be a photodetector selected to receive light in the range emitted from the emitter 18 after it has passed through the tissue. Additionally, the emitter 16 and the detector 18 may operate in various modes (e.g., reflectance or transmission). However, as noted above, the patient monitoring system 10 may be configured to determine a variety of physiological parameters with a suitable medical sensor. Accordingly, in certain embodiments, the sensor 14 may include sensing components in addition to, or instead of, the emitter 16 and the detector 18. For example, in one embodiment, the sensor 14 may include one or more electrodes (e.g., four electrodes) to obtain an electroencephalography signal.

The sensor 14 may be communicatively coupled to the patient monitor 12. In certain embodiments, the sensor 14 may include a wireless module configured to establish a wireless communication with the patient monitor 12 using any suitable wireless standard. For example, the sensor 14 may include a transceiver 20 that may enable wireless operation signals to be transmitted to and received from an external device (e.g., the patient monitor 12, a charging device, a disinfecting device, etc.). The transceiver 20 may establish wireless communication with a transceiver 22 of the patient monitor using any suitable protocol. For example, the transceiver 20 may be configured to transmit signals using one or more of the ZigBee standard, WirelessHART standard, Bluetooth standard, IEEE 802.11x standards, or MiWi standard. Additionally, the transceiver 20 may transmit a raw digitized detector signal, a processed digitized detector signal, and/or a calculated physiological parameter, as well as any data that may be stored in the sensor, such as data relating to a disinfection status of the sensor 10 and/or data relating to a disinfection process, as discussed below. Additionally or alternatively, the emitters 16 and detectors 18 of the sensor 14 may be coupled to the patient monitor 12 via a cable 24 through a plug 26 (e.g., a connector having one or more conductors) coupled to a sensor port. Thus, in certain embodiments, the sensor 14 may be configured to operate in both a wireless mode and a wired mode. Accordingly, in certain embodiments, the cable 24 may be removably attached to the sensor 14 via a connector 28 (e.g., a plug or a magnet), which may increase the patient's range of motion while wearing the sensor 14.

The patient monitor 12 may be configured to calculate physiological parameters of the patient relating to the physiological signal received from the sensor 14. For example, the patient monitor 12 may include a processor configured to calculate the patient's arterial blood oxygen saturation, pulse rate, respiration rate, blood pressure, blood pressure characteristic measure, a bispectral index, and/or any other suitable physiological characteristics. Additionally, the patient monitor 12 may include a monitor display 30 configured to display information regarding the physiological parameters, information about the system (e.g., instructions for disinfecting and/or charging the sensor 14), and/or alarm indications. The patient monitor 12 may include various input components 32, such as knobs, switches, keys and keypads, buttons, etc., to provide for operation and configuration of the patient monitor 12. The patient monitor 12 may also display information related to alarms, monitor settings, and/or signal quality via one or more indicator lights 33.

As noted above, the patient monitor 12 may be any suitable monitor, such as a pulse oximetry monitor. Furthermore, to upgrade the conventional operation provided by the patient monitor 12 and to provide additional functions, the patient monitor 12 may be coupled to a multi-parameter monitor 34 via a cable 36 connected to a sensor input port or via a cable 38 connected to a digital communication port. In addition to the patient monitor 12, or alternatively, the multi-parameter monitor 34 may be configured to calculate physiological parameters and to provide a central display 40 for the visualization of information from the patient monitor 12 and from other medical monitoring devices or systems. The multi-parameter monitor 34 includes a processor that may be configured to execute code for calculating one or more physiological parameters and displaying the physiological parameters and/or other information about the patient monitoring system 10. The multi-parameter monitor 34 may also include various input components 42, such as knobs, switches, keys and keypads, buttons, etc., to provide for operation and configuration of the multi-parameter monitor 34. In addition, the patient monitor 12, the multi-parameter monitor 34, and/or the sensor 14 may be connected to a network to enable the sharing of information with servers or other workstations.

Because the sensor 14 may be configured to operate in a wireless mode and, in certain embodiments, may not receive power from the patient monitor 12 while operating in the wireless mode, the sensor 14 may include a battery 44 to provide power to the components of the sensor 14 (e.g., the emitter 16 and the detector 18). In certain embodiments, the battery 44 may be a rechargeable battery such as, for example, a lithium ion, lithium polymer, nickel-metal hydride, or nickel-cadmium battery. However, any suitable power source may be utilized, such as, one or more capacitors and/or an energy harvesting power supply (e.g., a motion generated energy harvesting device, thermoelectric generated energy harvesting device, or similar devices).

The sensor 14 may also include a sensor body 46 to house the components of the sensor 14 (e.g., the emitter 16, the detector 18, the transceiver 20, and/or the battery 44). In certain embodiments, the sensor body 46 may be formed from any suitable material that can be disinfected and/or may be shaped to minimize or eliminate crevices to facilitate disinfecting of the sensor 14. In particular, the sensor body 46 may be resistant to or may prevent fluid infiltration. For example, the sensor body 46 may be formed from rigid or conformable materials, such as rubber or elastomeric compositions (including acrylic elastomers, polyimide, silicones, silicone rubber, celluloid, PMDS elastomer, polyurethane, polypropylene, acrylics, nitrile, PVC films, acetates, and latex). Further, the sensor 14 may be formed from molded or overmolded components. Additionally, the sensor body 46 may include a cover (e.g., a flap) that may be at least partially removed from the sensor body 46 and may cover and/or protect the connector 28 from a disinfecting agent, when the connector 28 is not coupled to the cable 24. In other embodiments, the sensor 14 may be disposable (e.g., a single-use sensor), and thus, the sensor body 46 may be constructed from any suitable materials, which may not be suitable for disinfecting (e.g., fabric or paper). Further, in other embodiments, the sensor 14 may be partially reusable. For example, the sensor body 46 may include an adhesive layer disposed on a patient-contacting surface of the sensor body 46 (e.g., to attach the sensor 14 to the patient's tissue), and the adhesive layer may be removed and disposed of prior to disinfecting the sensor 14.

Additionally, the sensor 10 may include one or more additional sensors 48 disposed on and/or in the sensor body 46. As will be discussed in more detail below, the one or more additional sensors 48 may generate information relating to a condition of the sensor 14, and in particular may generate information relating to changes in the condition of the sensor 14 during a disinfection process (e.g., autoclaving and/or exposure to a disinfecting solution and/or disinfecting gas). For example, the one or more additional sensors 48 may be pressure sensors, temperature sensors, moisture sensors, proximity detectors, or any other suitable sensor. The signals generated by the one or more additional sensors 48 may be utilized by the sensor 14 to determine whether the sensor 14 has been disinfected and, in some embodiments, whether the sensor 14 was properly disinfected (e.g., exposed to a predetermined pressure or temperature).

Turning to FIG. 2, a block diagram of the patient monitoring system 10 is illustrated in accordance with an embodiment. Specifically, certain components of the sensor 14 and the patient monitor 12 are illustrated in FIG. 2. As noted above, the sensor 14 may include the emitter 16 and the detector 18. The emitter 16 may emit light into a patient 60, which may be reflected by or transmitted through the patient 16 and subsequently detected by the detector 18. In some embodiments, the emitter 16 may emit one or more different wavelengths of light. For example, the emitter 16 may emit red wavelengths between approximately 600 nanometers (nm) and 700 nm and/or infrared wavelengths between approximately 800 nm and 1000 nm. In other embodiments, the emitter 16 may emit a red wavelength between approximately 620 nm and 700 nm (e.g., 660 nm), a far red wavelength between approximately 690 nm and 770 nm (e.g., 730 nm), and an infrared wavelength between approximately 860 nm and 940 nm (e.g., 900 nm). Other wavelengths may include, for example, wavelengths between approximately 500 nm and 600 nm and/or 1000 nm and 1100 nm. Alternative light sources may be used in other embodiments. For example, a single wide-spectrum light source may be used, and the detector 18 may be configured to detect certain wavelengths of light. In another example, the detector 18 may detect a wide spectrum of wavelengths of light, and the patient monitor 12 may process only those wavelengths which are of interest for use in measuring, for example, water fractions, hematocrit, or other physiologic parameters of the patient 60. It should be understood that, as used herein, the term “light” may refer to one or more of ultrasound, radio, microwave, millimeter wave, infrared, visible, ultraviolet, gamma ray, or X-ray electromagnetic radiation, and may also include any wavelength within the radio, microwave, millimeter wave, infrared, visible, ultraviolet, or X-ray spectra, and that any suitable wavelength of light may be appropriate for use with the present disclosure.

Additionally, the sensor 14 may include an encoder 62, which may contain information about the sensor 14. For example, the encoder 62 may contain information regarding what type of sensor it is (e.g., whether the sensor is intended for placement on a forehead or digit) and the wavelengths of light emitted by the emitter 16. This information may enable the patient monitor 12 to select appropriate algorithms and/or calibration coefficients for calculating the physiological characteristics of the patient 60. Additionally, the encoder 62 may include information relating to proper disinfecting of the sensor 14. For example, the encoder 62 may include information relating to the number of times the sensor 14 has been disinfected and/or the type of disinfecting agents (e.g., an autoclave, solutions, and/or gases) that may be used to disinfect the sensor 14. In one embodiment, the encoder 62 may store information relating to the type of sensor 14, such as, for example, whether the sensor 14 is reusable, partially reusable, or disposable. In some embodiments, the encoder 62 may also store information relating to a minimum pressure threshold and/or a minimum temperature threshold, or any other suitable threshold that the sensor 14 may be exposed to during a disinfecting process to determine whether the sensor 14 was properly disinfected during the disinfection process. The encoder 62 may also be programmed with information relating to the battery 44 such as, for example, the type of battery 44 utilized and an approximate use time for the battery 44 before the battery 44 may be drained. The encoder 62 may, for instance, be a memory, a coded resistor, EEPROM, or other coding devices that may provide a signal to the patient monitor 12 relating to the characteristics of the sensor 14. The patient monitor 12 may include a reader/decoder 64 that may read and/or decode information from the encoder 62 to provide the patient monitor 12 with information about the sensor 14.

In one implementation, signals from the detector 18 (and data from the encoder 62, if present) may be transmitted to the patient monitor 12 via the transceiver 20 and/or the cable 24. The patient monitor 12 may include data processing circuitry (such as one or more processors 66, application specific integrated circuits (ASICS), or so forth) coupled to an internal bus 68. Also connected to the bus 68 may be a RAM memory 70, a ROM memory 72, a speaker 74, the transceiver 22, the display 30, and/or the input components 32. In one embodiment, a time processing unit (TPU) 76 may provide timing control signals to light drive circuitry 78, which may control the operation of the emitter 16, such as to control when, for how long, and/or how frequently the emitter 16 is activated, and if multiple light sources are used, the multiplexed timing for the different light sources.

The TPU 76 may also control the gating-in of signals from the detector 18 through a switching circuit 80. These signals may be sampled at the proper time, depending upon which light source is illuminated. The received signal from the detector 18 may be passed through an amplifier 82, a low pass filter 88, and an analog-to-digital converter (A/D) 90 for amplifying, filtering, and digitizing the signals from the sensor 14. The digital data may then be stored in a queued serial module (QSM) 92 for later downloading to the RAM 70 as the QSM 92 fills up. In an embodiment, there may be multiple parallel paths of separate amplifier, filter, and A/D converters for multiple wavelengths or spectra received.

In some embodiments, in addition to or instead of the patient monitor 12, the sensor 14 may be configured to control the acquisition of the physiological signal, to process the physiological signal, and/or to calculate one or more physiological parameters based upon the physiological signal. This may be advantageous in certain scenarios in which the sensor 14 is moved outside of the wireless range of the patient monitor 12 (e.g., a patient steps outside of the room) and sensor operation is still desired. In such embodiments, the sensor 14 may include a light drive 98 configured to drive the emitter 16 and the detector 18 based upon control signals based upon control signals from a TPU 100 of the sensor 14. The light drive 98 may also be configured to operate based upon control signals received from the patient monitor 12 via the transceiver 20 (e.g., when the sensor 14 is within the wireless range of the patient monitor 12). The TPU 100 may operate in a similar manner as the TPU 76. The received signal from the detector 18 may be passed through an amplifier 102, a low pass filter 104, and an analog-to-digital converter 106. The digital data may then be stored in a non-volatile (NV) memory 108, which may be coupled to the main system bus 110. Additionally, the NV memory 108 may also store historical data and/or values (e.g., detector signal data, data points, trend information, etc.) for the physiological parameter of the patient. In certain embodiments, as will be described in more detail below, the sensor 14 may be configured to store raw digital data and/or minimally processed data in the NV memory 108 for transmitting to the patient monitor 12 for further processing and/or calculating of physiological parameters when the sensor 14 is placed in operational proximity of the patient monitor 12. In this manner, the sensor 14 may minimize power consumption while operating wirelessly. Additionally, in certain embodiments, the sensor 14 may also include data processing circuitry (such as one or more processors 112, application specific integrated circuits (ASICS), or so forth) coupled to the internal bus 110 for additional signal processing and/or calculating of physiological parameters.

Regardless of the degree of processing performed by the sensor 14, in certain embodiments, based at least in part upon the received signals corresponding to the light received by the detector 18, the processor 66 may calculate physiological characteristics of the patient 60, such as the oxygen saturation of the patient 60, using various algorithms. These algorithms may use coefficients, which may be empirically determined, and may correspond to the wavelengths of light used. The algorithms may be stored in the ROM 72 and accessed and operated according to processor 66 instructions. As noted above, the patient monitor 12 may also receive information from the encoder 62, which may be decoded by the decoder 64 and provided to the processor 66. In particular, the decoded signals may provide information to the processor 66 such as the sensor type and the wavelengths of light emitted by the emitter 16, so that proper calibration coefficients and/or algorithms to be used for calculating physiological characteristics of the patient 60 may be selected and utilized by the processor 66.

As noted above, the sensor 14 may also include one or more additional sensors 48, which may be configured to generate information relating to a condition of the sensor 14. For example, the one or more additional sensors 48 may include temperature sensors, pressure sensors, moisture sensors, proximity detectors, ultrasonic detectors, colorant bacterial indicators, or any other suitable sensor configured to provide information relating to a condition of the sensor 14. In certain embodiments, the sensor 14 may be configured to transmit one or more signals generated by the one or more additional sensors 48 to the patient monitor 12 for processing and/or the calculation of one or more parameters as described herein. In other embodiments, the processor 112 and/or other data processing circuitry of the sensor 14 may be configured to process the one or more signals and to calculate the one or more parameters based on the signals.

In particular, the processor 112 may utilize signals generated by the additional sensors 48 to determine a disinfection condition of the sensor 14. As noted above, a first disinfection condition of the sensor 14 may indicate that continued sensor operation is acceptable, and a second disinfection condition may indicate that disinfecting the sensor 14 (if reusable) or disposing of the sensor 14 (if disposable) may be desirable. The processor 112 may also utilize signals generated by the additional sensors 48 to monitor changes in a condition (e.g., pressure, temperature, etc.) of the sensor 14 during a disinfecting process, which will be described in more detail below with respect to FIG. 4. However, it should be noted that in some embodiments, the sensor 14 may include data processing circuitry configured to determine the disinfection condition of the sensor 14 and/or to monitor changes in the condition of the sensor 14 during a disinfection process, rather than the processor 112.

For example, in certain embodiments, the additional sensors 48 may include one or more colorant bacterial indicators that may be configured to change colors in the presence of various bacteria. The colorant bacterial indicators may, for example, include a substrate and/or absorbent pad impregnated with a solution that may undergo a color change in the presence of the bacteria. A color change may occur due to a change in the pH of the solution and/or an increase in carbon dioxide and/or other by-products of bacterial growth in the solution. By way of example, the solution may include a solvatochromic dye and/or a pH indicator. The resulting color change may be visually detected by a user; however, the user may experience difficulty in discerning subtle gradations of the color change, which may provide information relating to the measure (e.g., grade or degree) of disinfection and may be useful in determining whether it may be desirable to disinfect the sensor 14.

Accordingly, in certain embodiments, the patient monitoring system 10 may include an optical scanner 120, such as a hand-held scanner, which may be configured to scan the colorant bacterial indicator and generate a signal based at least in part upon the color of the colorant bacterial indicator. The optical scanner 120 may transmit the signal to the sensor 14 and/or the patient monitor 12 via a transceiver 122 or a wired connection for further processing. In one embodiment, the processor 112 of the sensor 14 may determine a measure of disinfection of the sensor 14 based on the signal received from the optical scanner 120 and may compare the measure of disinfection to a predetermined threshold for disinfection. The processor 112 may determine that the sensor 14 may correspond to the second disinfection condition, in which disinfection and/or disposal may be desirable, in response to determining that the measure of disinfection exceeds the predetermined threshold. Additionally, the sensor 14 may provide a user-perceivable indication of the disinfection condition (e.g., the second disinfection condition). The user-perceivable indication may be a visual indication via one of the one or more indicator lights 124, which may alert the user that it may be desirable to disinfect the sensor 14. It should be noted that any suitable indication may be provided, such as an audible indication via a speaker and/or a graphical or textual indication via a display. In other embodiments, the optical scanner 120 and/or the patient monitor 12 may be configured to determine the measure of disinfection of the sensor 14, to determine whether the measure of disinfection of the sensor 14 exceeds the predetermined threshold for disinfection, and/or to provide the user-perceivable indication.

Additionally or alternatively, the processor 112 of the sensor 14 may be configured to determine a disinfection condition of the sensor 14 by determining an amount of time that has elapsed since the sensor 14 was last disinfected and/or since the sensor 14 has been in use. For example, for embodiments in which the sensor 14 is disposable, the processor 112 may be configured to determine an amount of time that the sensor 14 has been in operation and to compare the amount of time to a predetermined threshold for sensor operation. The processor 112 may determine that the sensor 14 may correspond to the second disinfection condition, in which disposing of the sensor 14 may be desirable, in response to determining that the elapsed time exceeds the predetermined threshold. The sensor 14 may provide a user-perceivable indication, such as emitting light from one or more of the indicator lights 124, in response to determining that the amount of time exceeds the predetermined threshold for sensor operation. This indication may alert the user that it may be desirable to dispose of the sensor 14.

Additionally, for embodiments in which the sensor 14 is at least partially reusable, the processor 112 may be configured to determine an amount of time that has elapsed since the sensor 14 was last disinfected or an amount of time that the sensor 14 has been in use, if the sensor 14 has not been disinfected. Accordingly, as will be described in more detail below, the NV memory 108 may be configured to store information relating to prior disinfection processes, and the processor 112 may be configured to access the NV memory 108 to obtain time information for the last disinfection process, which may be used to determine when the sensor 14 was last disinfected. The processor 112 may compare the amount of time to a predetermined threshold for sensor operation, and may determine that the sensor 14 corresponds to the second disinfection condition, in which disinfecting the sensor 14 may be desirable, in response to determining that the elapsed time exceeds the predetermined threshold for sensor operation. In certain embodiments, the sensor 14 may provide a user-perceivable indication, such as emitting light from one or more of the indicator lights 124, in response to determining that the amount of time exceeds the predetermined threshold for sensor operation. This indication may alert the user that it may be desirable to disinfect the sensor 14.

As described above, the patient monitoring system 10 may be configured to determine a disinfection condition of the sensor 14 and to provide an indication to a user that it may be desirable to disinfect the sensor 14 when the disinfection condition of the sensor 14 exceeds a respective threshold. The present embodiments also provide various methods for providing an indication to disinfect the sensor 14 in accordance with the embodiments discussed above. For example, FIG. 3 illustrates an embodiment of a method 150 for providing an indication to disinfect reusable sensors 14 and for providing an indication to dispose of single-use sensors 14. Certain steps of the method 150 may be performed by a processor, or a processor-based device, such as the sensor 14 and/or the patient monitor 12, which may each include instructions for implementing certain steps of the method 150. Additionally, the instructions for implementing certain steps of the method 150 may be stored as coded instructions and/or algorithms in a memory device (e.g., the NV memory 108, the RAM 70, and/or the ROM 72) and may be accessed and executed by a processor (e.g., the processor 112 and/or the processor 66).

The method 150 includes operating the sensor 14 (block 152). As described in detail above, operating the sensor (block 152) may include driving the emitter 16 and detecting light after it has passed through the patient's tissue using the detector 18. For embodiments in which the sensor 14 is at least partially reusable, the method 150 may include determining an elapsed time since the sensor 14 was last disinfected (block 154). In certain embodiments, determining the elapsed time (block 154) may include accessing the NV memory 108 to determine when the sensor 14 was last disinfected, which may be stored, for example, as a timestamp for a completed disinfection process. In other embodiments, a timestamp for the last disinfection process of the sensor 14 may be stored in the RAM 70 and/or the ROM 72 of the patient monitor 12. The processor-based device (e.g., the sensor 14 and/or the patient monitor 12) may compare the determined elapsed time to a predetermined time threshold to determine whether the elapsed time is less than the predetermined time threshold (block 156). The predetermined time threshold may be a predetermined acceptable amount of time that the sensor 14 may operate before disinfection may be desirable. In certain embodiments, the predetermined time threshold may be between approximately 30 minutes and 48 hours, 1 hour and 36 hours, 2 hours and 24 hours, 3 hours and 12 hours, 4 hours and 10 hours, or any other time range. If the elapsed time is less than the predetermined time threshold, the method 150 may continue sensor operation (block 152).

Alternatively, in response to determining that the elapsed time exceeds the predetermined time threshold, the processor-based device (e.g., the sensor 14 and/or the patient monitor 12) may be configured to determine how many times the sensor 14 has been disinfected (block 158). Certain reusable sensors 14 may have a maximum number of times they may be disinfected before it may be desirable to dispose of the sensor 14. Accordingly, the method 150 may include determining whether the number of times the sensor 14 has been disinfected is less than a predetermined threshold for disinfections (block 160). The predetermined threshold for disinfections may be between approximately 1 and 100 disinfections, 10 and 90 disinfections, 20 and 80 disinfections, 30 and 70 disinfections, 40 and 60 disinfections, or any other range. In response to determining that the number of times the sensor 14 has been disinfected is less than the predetermined threshold for disinfections, the processor-based device (e.g., the sensor 14 and/or the patient monitor 12) may provide an indication to disinfect the sensor 14 (block 162). The indication may be a visual indication, such as a symbol and/or a textual message on a display (e.g., the display 30 of the patient monitor 12 or a display of the sensor 14), or a constant or flashing light (e.g., via the indicator lights 124). The indication may also be an auditory indication, such as a beep. Further, in certain embodiments, the indication to disinfect the sensor 14 (block 162) may be generated at a remote monitoring station. For example, the processor-based device (e.g., the sensor 14 and/or the patient monitor 12) may be configured to transmit a signal to the remote monitoring station (e.g., via a central server), which may cause the remote monitoring station to provide the indication to disinfect the sensor 14 (block 162).

Alternatively, if the number of times the sensor 14 has been disinfected is greater than the predetermined threshold for disinfections, the processor-based device (e.g., the sensor 14 and/or the patient monitor 12) may provide an indication to dispose of the sensor 14 (block 164). The indication may be a visual indication, such as a symbol and/or a textual message on a display (e.g., the display 30 of the patient monitor 12 or a display of the sensor 14), or a constant or flashing light (e.g., via the indicator lights 124). The indication may also be an auditory indication, such as a beep. Further, in certain embodiments, the indication to dispose of the sensor 14 (block 164) may be generated at a remote monitoring station. For example, the processor-based device (e.g., the sensor 14 and/or the patient monitor 12) may be configured to transmit a signal to the remote monitoring station (e.g., via a central server), which may cause the remote monitoring station to provide the indication to dispose of the sensor 14 (block 164). Additionally, in certain embodiments, the indication to dispose of the sensor 14 (block 164) may be provided as a different indication from the indication to disinfect the sensor 14 (block 162). For example, in certain embodiments, the sensor 14 may include at least two indicator lights 124, and the processor 112 may be configured to cause one of the indicator lights 124 to emit light (e.g., a first color) for the indication to disinfect the sensor 14 (block 162) and may cause another of the indicator lights 124 to emit light (e.g., a second color) for the indication to dispose of the sensor 14 (block 164). In one embodiment, a single indication light 124 may be utilized that may emit light of different colors.

In some embodiments, the method 150 may also include determining a measure of disinfection of the sensor 14 (block 166). In certain embodiments, determining a measure of disinfection of the sensor 14 (block 166) may include receiving one or more signals generated by the optical scanner 120 in response to scanning a colorant bacterial indicator disposed on the sensor body 46. As described in detail above, the colorant bacterial indicator may be configured to undergo a color change in the presence of bacteria, which may be detected by the optical scanner 120. In certain embodiments, the optical scanner 120 may determine the measure of disinfection and may transmit the measure of disinfection to the sensor 14 and/or the patient monitor 12. In other embodiments, the optical scanner 120 may transmit a raw or a partially-processed signal to the sensor 14 and/or the patient monitor 12 for further processing and the calculation of the measure of disinfection. The processor-based device (e.g., the sensor 14 and/or the patient monitor 12) may determine whether the measure of disinfection is less than a predetermined disinfection threshold (block 168). If the elapsed time is less than the predetermined time threshold, the method 150 may continue sensor operation (block 152). Alternatively, if the elapsed time exceeds the predetermined time threshold, the processor-based device (e.g., the sensor 14 and/or the patient monitor 12) may be configured to determine whether the sensor 14 is reusable (block 170). Determining whether the sensor is reusable (block 170) may be desirable, because it may determine whether the provided indication should alert the user to disinfect or dispose of the sensor 14. In some embodiments, the processor-based device (e.g., the sensor 14 and/or the patient monitor 12) may access and read stored information from the encoder 62 to determine whether the sensor 14 is reusable.

In response to determining that the sensor 14 is reusable, the processor-based device (e.g., the sensor 14 and/or the patient monitor 12) may provide the indication to disinfect the sensor 14 (block 162). Alternatively, if the sensor 14 is disposable, the processor-based device (e.g., the sensor 14 and/or the patient monitor 12) may be configured to provide the indication to dispose of the sensor 14 (block 164).

The method 150 may also include determining an elapsed time since the sensor 14 first began operating (block 172). Determining the elapsed time since the sensor 14 first began operating (block 172) may be desirable for disposable (e.g., single use) sensors 14, which may have a maximum operating time before disposal may be desirable. In certain embodiments, determining the elapsed time since the sensor 14 first began operating (block 172) may include accessing time data from the processor 112 and/or a clock (e.g., a real-time clock) of the sensor 14. Accordingly, the processor-based device (e.g., the sensor 14 and/or the patient monitor 12) may be configured to determine whether the elapsed time is less than a predetermined time threshold for sensor operation (block 174). In certain embodiments, the predetermined time threshold may be between approximately 30 minutes and 48 hours, 1 hour and 36 hours, 2 hours and 24 hours, 3 hours and 12 hours, 4 hours and 10 hours, or any other time range. If the elapsed time is less than the predetermined time threshold, the method 150 may continue sensor operation (block 152). In response to determining that the elapsed time is greater than the predetermined time threshold for sensor operation, the processor-based device (e.g., the sensor 14 and/or the patient monitor 12) may be configured to provide the indication to dispose of the sensor 14 (block 164).

Accordingly, when it is determined that it may be desirable to disinfect the sensor 14, the sensor 14 may be provided to a disinfecting device. For example, FIG. 4 illustrates a disinfecting system 190 that may be used to disinfect the sensor 14. As illustrated, the disinfecting system 190 includes a disinfecting unit 192 that may be configured to disinfect the sensor 14. In certain embodiments, the disinfecting unit 192 may include one or more disinfecting devices 194, which may be different types, such as, for example, an ultrasonic device 196, a disinfecting solution device 198, a disinfecting gas device 200, an autoclave 202, and a radiation (e.g., UV radiation and/or ionizing radiation) device 204. However, it should be noted, that in certain embodiments, the disinfecting unit 192 may include one, two, three, four, or any number of disinfecting devices 194. Additionally, the disinfecting system 190 may include any other suitable type of device for disinfecting the sensor 14. Furthermore, while the disinfecting devices 194 are illustrated as disposed in a single housing 205 of the disinfecting unit 192, in other embodiments, the disinfecting system 190 may include disinfecting devices 194 that are separate from one another (e.g., disposed in separate housings).

In certain embodiments, the sensor 14 may be manually placed into one or more of the disinfecting devices 194. In one embodiment, the disinfecting unit 192 may be constructed as an automatic multi-stage disinfecting process in which the sensor 14 is transferred between disinfecting devices 194 (e.g., after the completion of a disinfection process in one disinfecting device 194). By way of example, the disinfecting unit 192 may include a conveyor belt, and one or more of the walls of the disinfecting devices 194 may be configured to at least partially move and/or open to enable the sensor 14 to move between disinfecting devices 194. However, in another embodiment, the disinfecting unit 192 may be constructed to enable more than one disinfecting process to occur within the housing 205. That is, the disinfecting unit 192 may include components of two or more of the disinfecting devices 194, and may be configured to implement at least two different disinfecting processes either simultaneously or in a predetermined order. In certain embodiments, the disinfecting unit 192 and/or each disinfecting device 194 may include a processor 206 configured to control the operation of the disinfecting unit 192 or the disinfecting device 194, respectively.

Regardless of the type or number of disinfecting devices 194 that are utilized to disinfect the sensor 14, it may be desirable to determine whether the sensor 14 has been disinfected, what type of disinfecting device 194 (or disinfecting devices 194) were utilized to disinfect the sensor 14, and/or whether the disinfecting processes implemented by the one or more disinfecting devices 194 were successful (e.g., if the parameters for the disinfecting processes were within an acceptable range to enable disinfection of the sensor 14). Indeed, as noted above, the processor 112 of the sensor 14 may utilize signals generated by the additional sensors 48 to monitor changes in a condition (e.g., temperature, pressure, moisture, etc.) of the sensor 14 during one or more disinfection processes, which may provide information regarding the type of disinfection devices 194 utilized, the duration of time the sensor 14 was exposed to each disinfecting process, and/or the quality of each disinfection process (e.g., successful or faulty). Specifically, the one or more additional sensors 48 may generate signals, or combinations of signals, which may be identified by the processor 112 as corresponding to a particular disinfecting process. By way of example, the sensor 14 may include a temperature sensor and a pressure sensor, which may provide signals to the processor 112 indicating that the sensor 14 was exposed to high temperatures and high pressures, and the processor 112 may determine that the sensor 14 was disinfected using an autoclave. Various embodiments of the additional sensors 48 will be described in detail below and, in particular, will be discussed in combination with various types of the disinfecting devices 194.

The one or more signals from the additional sensors 48 and/or any information determined by the processor 112 based upon the one or more signals may be stored in the NV memory 108. The NV memory 108 and/or the encoder 62, if utilized, may also be configured to store predetermined thresholds for one or more parameters (e.g., pressure, temperature, moisture, time) for one or more disinfecting processes (e.g., ultrasonic cleaning, exposure to a disinfecting solution and/or gas, autoclaving, exposure to radiation). As will be described in more detail below with respect to FIG. 5, the predetermined thresholds may be utilized by the processor 112 to determine whether a particular disinfecting process may have been successful in disinfecting the sensor 14. The various predetermined thresholds will be described below with respect to their corresponding disinfecting processes. Further, it should be noted that the predetermined threshold may vary based upon a particular sensor type (e.g., clip-style sensor, adhesive sensor, adult sensor, pediatric sensor, neonatal sensor, etc.).

However, in addition to the additional sensors 48, the sensor 14 may also be configured to receive wireless signals via the transceiver 20, which may provide information for determining whether the sensor 14 was disinfected and/or what type of disinfecting devices 194 were utilized to disinfect the sensor 14. In particular, the disinfecting unit 192 may include at least one transceiver 208, which may be configured to establish wireless communication with the transceiver 20 using any suitable protocol. In some embodiments, each disinfecting device 194 of the disinfecting unit may include the transceiver 208. In some embodiments, the sensor 14 may be configured to transmit an interrogation signal via the transceiver 20, which may be received by the one or more transceivers 208 when the sensor 14 is in operational proximity of the respective disinfecting device 194. As used herein, operation proximity is defined as a distance between the transceiver 20 of the sensor 14 and the transceiver 206 of one of the disinfecting devices 194 that enables wireless signals to be transmitted and received between the transceiver 20 and the transceiver 208. Additionally, the one or more transceivers 208 may each transmit a confirmation signal to the sensor 14 in response to receiving the interrogation signal. In other embodiments, the one or more transceivers 208 may be configured to at least periodically transmit interrogation signals to the sensor 14, which may be received by the transceiver 20 when the sensor 14 is in operational proximity to the respective disinfecting device 194. In certain embodiments, the processor 112 may be configured to determine that the sensor 14 is disposed within one of the disinfecting devices 194 based at least in part upon a received signal from the transceiver 208 of the respective disinfecting device 194. In some embodiments, the transceiver 208 may also transmit one or more signals to the sensor 14 indicating that the disinfecting device 194 is disinfecting the sensor 14 and/or that the disinfecting device 194 has completed the disinfecting process for disinfecting the sensor 14. This may be advantageous to minimize any false determinations of a completed disinfection by the sensor 14, which may occur, for example, if the sensor 14 is placed in the disinfecting unit 192 without operating the disinfecting unit 192.

Additionally or alternatively, the sensor 14 may include a radio-frequency identification (RFID) tag 210. The RFID tag 210 may be an active tag, which may transmit radio-frequency signals to an RFID antenna unit (e.g., an RFID antenna unit 212 of the disinfecting unit 192). Alternatively, the RFID tag 210 may be a passive tag, which may be activated and powered by a signal transmitted from the RFID antenna unit (e.g., the RFID antenna unit 212). In some embodiments, the disinfecting unit 192 may include the RFID antenna unit 212 in each disinfecting device 194. This may be advantageous, because the RFID antenna units 212 may detect the RFID tag 210 (e.g., may “read” the sensor 14) in each disinfecting unit 194. In this manner, the sensor 14 may be tracked through each stage of the disinfecting unit 192. In certain embodiments, the disinfecting unit 192 may be configured to store information related to the tracking of the sensor 14 in a memory device (not shown). Additionally, the disinfecting unit 192 may be configured to transmit the information relating to the tracking to the sensor 14 and/or a remote device (e.g., the patient monitor 12 and/or a remote monitoring station). The disinfecting unit 192 may be configured to transmit the information wirelessly and/or through a wired connection.

As noted above, the disinfecting unit 192 may include the ultrasonic device 196. The ultrasonic device 196 may include a transducer 214 configured to generate ultrasonic waves through a liquid (e.g., water, a cleaning solvent, or a disinfecting solution) disposed in the ultrasonic device 196. The ultrasonic device 196 may be desirable to loosen and/or remove debris from the sensor 14. In certain embodiments, the minimum predetermined time threshold for exposure to the ultrasonic waves may be between approximately 1 second to 10 minutes, 5 seconds to 5 minutes, 10 seconds to 3 minutes, 20 seconds to 2 minutes, or any suitable time range. As illustrated, the sensor 14 may include the one or more additional sensors 48, which may include an ultrasonic pressure sensor 216 configured to detect the ultrasonic waves.

The disinfecting unit 192 may also include the disinfecting solution device 198. The disinfecting solution device 198 may utilize any suitable solution or combinations of solutions for disinfecting the sensor 14, such as alcohols, aldehydes, phenolics, halogens, heavy metals, surface active agents, and/or dyes. In certain embodiments, the disinfecting solution device 198 may utilize a solution of approximately 70 percent isopropyl alcohol and/or a 1:10 bleach solution. The disinfection solution device 198 may be filled with one or more disinfecting solutions either manually or automatically. Additionally or alternatively, the disinfection solution device 198 may include one or more sprayers 218 configured to spray the one or more disinfecting solutions on the sensor 14. In certain embodiments, the minimum predetermined time threshold for exposure to the disinfecting solution may be between approximately 1 minute and 2 hours, 5 minutes and 1.5 hours, 10 minutes and 1 hour, 20 minutes and 45 minutes, or any other time range. As illustrated, the sensor 14 may include the one or more additional sensors 48, which may include a moisture sensor 220 configured to detect exposure to a disinfecting solution and/or a pressure sensor 222 configured to detect pressure changes from contact due to the disinfecting solution (e.g., from the sprayers 218).

In certain embodiments, the disinfecting unit 192 may also include the disinfecting gas device 200. The disinfecting gas device 200 may utilize any suitable gas or combination of gases for disinfecting the sensor 14. For example, the disinfecting gas device 200 may utilize formaldehyde, plasma, chlorine dioxide, and/or ethylene oxide. The disinfecting gas device 200 may include one or more valves (not shown) to enable the one or more disinfecting gases to enter the disinfecting gas device 200 (e.g., from a reservoir). In some embodiments, the disinfecting gas device 200 may also include one or more sprayers 226 configured to spray the disinfecting gas on the sensor 14. Additionally, in one embodiment, the disinfecting gas device 200 may include one or more aerators (not shown). The one or more aerators may be utilized to dissipate any remaining disinfecting gas from the sensor 14 and may be particularly advantageous when utilizing ethylene oxide. In certain embodiments, the sensor 14 may include the pressure sensor 222, which may be configured to detect pressure changes from contact due to the disinfecting gas (e.g., from the sprayers 226). In one embodiment, the sensor 14 may include one or more gas sensors (not shown) configured to detect one or more types of gas.

Additionally, in certain embodiments, the disinfecting gas device 200 may also be configured for dry heat sterilization. For example, the disinfecting gas device 200 may include a heat source 230 to provide heat to the disinfecting gas device 200. Additionally, in one embodiment, the disinfecting gas device 200 may be configured to utilize air, rather than a disinfecting gas, in combination with the heat source 230 to implement a dry heat sterilization process. In certain embodiments, the minimum predetermined temperature threshold may be between approximately 10 degrees Celsius and 300 degrees Celsius, 50 degrees Celsius and 250 degrees Celsius, 100 degrees Celsius and 200 degrees Celsius, or 150 degrees Celsius and 180 degrees Celsius, or any other temperature range. Additionally, the minimum predetermined time threshold for exposure to the disinfecting gas and/or the heat may be between approximately 30 minutes and 3 hours, 45 minutes and 2.5 hours, 1 hour and 2 hour, or any other time range. Accordingly, in certain embodiments, the sensor 14 may include the one or more additional sensors 48, which may include a temperature sensor 232 configured to determine a temperature the sensor 14 is exposed to in the disinfecting gas device 200.

The disinfecting unit 192 may also include the autoclave 202 to disinfect the sensor 14. The autoclave 202 may be configured to provide steam at a high temperature and a high pressure. The minimum predetermined pressure threshold may be between approximately 30 kilopascals (kPa) to 280 kPa, 70 kPa to 210 kPa, 100 kPa to 170 kPa, or any other suitable pressure range. The minimum predetermined temperature threshold may be between approximately 100 degrees Celsius and 200 degrees Celsius, 110 degrees Celsius and 190 degrees Celsius, 120 degrees Celsius and 180 degrees Celsius, or any other suitable temperature range. Additionally, the minimum predetermined threshold for the period of time that the sensor 14 is exposed to the temperature and pressure may be between approximately 1 minute and 1 hour, 5 minutes and 50 minutes, 10 minutes and 40 minutes, 15 minutes and 30 minutes, or any other suitable range. In certain embodiments, the sensor 14 may include the pressure sensor 222 and the temperature sensor 232 to detect pressures and temperatures the sensor 14 is exposed to in the autoclave 202, respectively.

Additionally, in one embodiment, the disinfecting unit 192 may include the radiation device 204. The radiation device 204 may include a radiation source 234, which may be configured to direct radiation on the sensor 14 when the sensor 14 is disposed in the radiation device 204. In certain embodiments, the radiation source 234 may be configured to emit ultraviolet (UV) radiation and/or ionizing radiation, such as, for example, cathode rays, X-rays, electron-rays, and/or gamma-rays. The minimum predetermined time threshold for exposure to the radiation source 234 may be between approximately 1 second and 5 minutes, 5 seconds and 4 minutes, 10 seconds and 3 minutes, 20 seconds and 2 minutes, 30 seconds and 1 minute, or any other time range. In certain embodiments, the sensor 14 may include the additional sensors 48, which may include a semiconductor 236 (e.g., a silicon diode or a photodiode) configured to detect ionizing radiation. The semiconductor 236 may also be configured to detect UV radiation.

As described above, the processor 112 of the sensor 14 may be configured to receive signals from the additional sensors 48 (e.g., the ultrasonic pressure sensor 216, the moisture sensor 220, the pressure sensor 222, the temperature sensor 232, the semiconductor 236, and any other suitable sensor), signals from the transceiver 20 that may be transmitted from the transceiver 208 of the disinfecting unit 192, and/or signals from the RFID tag 210 and/or the RFID antenna unit 212. Based at least in part upon the received signals, the processor 112 may be configured to determine whether the sensor 14 was disinfected and/or which disinfecting device 194 or disinfecting devices 194 were utilized to disinfect the sensor 14. As will be described in detail below with respect to FIG. 5, the processor 112 may also utilize the signals to determine whether the disinfection processes met or exceeded minimum predetermined thresholds (e.g., to determine the quality of the disinfection processes). In some embodiments, the processor 112 may cause the sensor 14 to provide one or more user-perceivable indications of the disinfection process (or disinfection processes). By way of example, the processor 112 may cause one of the indicator lights 124 to emit light in response to determining that the sensor 14 has been disinfected and is ready for use and may cause another of the indicator lights 124 to emit light in response to determining that the disinfecting process utilized did not meet a minimum predetermined threshold. Additionally, the sensor 14 may be configured to store information relating to the disinfecting in the NV memory 108. This stored information may be transmitted to an external device (e.g., the patient monitor 12, the disinfecting unit 192, a remote monitoring station, or any other suitable device) via the transceiver 20 or a wired connection (e.g., the cable 24).

To determine quality information for the disinfection processes, the processor 112 may be configured to compare the one or more signals received from the additional sensors 48 to their respective minimum predetermined thresholds for each disinfection process, as described in detail above. In particular, the processor 112 may determine that the sensor 14 was properly disinfected if each relevant parameter (e.g., pressure, temperature, time, moisture, etc.) for a particular disinfection process (e.g., autoclaving, dry-heat sterilization, exposure to a disinfecting solution, exposure to a disinfecting gas, ultrasonic cleaning, exposure to UV and/or ionizing rays) is within an acceptable range for the given parameter (e.g., above the respective minimum predetermined threshold). For example, FIG. 5 illustrates an embodiment of a method 250 for providing an indication of a successful disinfection process utilized to disinfect the sensor 14. Certain steps of the method 250 may be performed by a processor, or a processor-based device, such as the sensor 14 and/or the patient monitor 12, which may each include instructions for implementing certain steps of the method 250. Additionally, the instructions for implementing certain steps of the method 250 may be stored as coded instructions and/or algorithms in a memory device (e.g., the NV memory 108, the RAM 70, and/or the ROM 72) and may be accessed and executed by a processor (e.g., the processor 112 and/or the processor 66).

The method 250 may include receiving one or more signals from one or more sensors (e.g., the additional sensors 48) disposed in or on a medical sensor (e.g., the sensor 14) exposed to a disinfection process (block 252). As described in detail above, the additional sensors 48 may include the ultrasonic pressure sensor 216, the moisture sensor 220, the pressure sensor 222, the temperature sensor 224, the semiconductor 234, and any other suitable sensor. The method 250 may also include receiving one or more signals from a disinfecting device (e.g., the disinfecting unit 192) implementing the disinfection process (block 254). As described in detail above, the one or more signals may be transmitted from the transceiver 208 of the disinfecting unit 192, which may inform the processor 112 that the sensor 14 is disposed in and/or is being disinfecting by one of the disinfecting devices 194 of the disinfecting unit 192. In some embodiments, the one or more signals may provide information to the processor 112 regarding the particular disinfecting process utilized. Additionally, the one or more signals may be generated by the RFID antenna unit 212 and transmitted to the sensor 14, which may provide information to the processor 112 regarding the tracking of the sensor 14 through the disinfecting devices 194 of the disinfecting unit 192.

The method 250 may include determining one or more parameters of the disinfection process based at least in part upon the received signals from the additional sensors 48 and/or the disinfecting unit 192 (block 256). As described in detail above, the one or more parameters may include pressure, temperature, time, and/or exposure to a disinfecting solution, a disinfecting gas, UV radiation, ionizing radiation, and/or ultrasonic waves. The processor-based device (e.g., the sensor 14 and/or the patient monitor 12) may be configured to determine the type of disinfection process (block 258) based at least in part upon the received signals from the additional sensors 48, the received signals from the disinfecting unit 192, and/or the determined parameters of the disinfection process. As described in detail above, the sensor 14 may have a different minimum predetermined threshold, which may be dependent upon the disinfection processes utilized to disinfect the sensor 14. Accordingly, it may be desirable to determine the disinfection process utilized to disinfect the sensor 14 to select the appropriate minimum predetermined threshold or thresholds.

Accordingly, the method 250 may include determining whether one or more determined parameters of the determined disinfection process are each above their respective minimum predetermined thresholds (block 260). In particular, the processor-based device (e.g., the sensor 14 and/or the patient monitor 12) may be configured to access the minimum predetermined thresholds stored in the NV memory 108, as described in detail above with respect to FIG. 4. The processor-based device (e.g., the sensor 14 and/or the patient monitor 12) may determine that the sensor 14 has been disinfected properly and may provide a user-perceivable indication of a successful disinfection process (block 262) in response to determining that the one or more determined parameters of the determined disinfection process are greater than or equal to the respective minimum predetermined thresholds. In one embodiment, the sensor 14 may be configured to provide the user-perceivable indication of a successful disinfection process (block 262), which may be an audible indication or a visual indication. For example, the processor 112 may cause one of the one or more indicator lights 124 to emit light. Additionally, in response to determining that one or more of the determined parameters are not above their respective minimum predetermined thresholds, the processor-based device (e.g., the sensor 14 and/or the patient monitor 12) may provide a user-perceivable indication of an unsuccessful disinfection process (block 264). In certain embodiments, the processor 112 may cause another of the indicator lights 124 to emit light, which may be of a different color than the indicator light 124 that is emitted when the processor 112 determines that the disinfection process was successful. In one embodiment, a single indicator light 124 may be configured to emit multiple colors. The user-perceivable indication of the unsuccessful disinfection process (block 264) may be provided as an alarm, which may be an audible alarm, such as a beep, and/or a textual alarm, such as an error message.

As noted above, monitoring the disinfection status of the sensor 14 and monitoring one or more changes in a condition of the sensor 14 during a disinfection process may increase the power consumption of the sensor 14. As a result, for embodiments in which the sensor 14 utilizes the battery 44 as a power source, problems may exist in which the battery 44 may drain, and the battery 44 must be recharged or replaced to continue sensor operation. Additionally, as noted above, the sensor 14 may be configured to operate using both power from the battery 44 and wired power (e.g., via the cable 24) from a charging device. In particular, the sensor 14 may operate using wired power when the sensor 14 is coupled to a charging device via the cable 24 and may operate using battery power when the sensor 14 is not coupled to a charging device. Accordingly, to minimize battery drainage, it may be desirable to minimize battery use and operate using wired power when the sensor 14 is within range of a charging device. Unfortunately, the user and/or the patient may forget to attach the sensor 14 to the cable (e.g., the cable 24) of the charging device when the sensor 14 is within range of the charging device.

To address this issue, the present embodiments describe techniques to detect when the sensor 14 is within a predetermined range of a charging device (e.g., the sensor 14 is located less than a minimum predetermined distance from the charging device) and to provide an indication to a user that the sensor 14 is within the predetermined range. As used herein, the sensor 14 may be in proximity to a charging device, if the sensor 14 is within the predetermined range of the charging device. For example, FIG. 6 illustrates a block diagram of a system 280 that may include the sensor 14 and a charging device 282 for providing power to the sensor 14 when the sensor 14 is physically coupled to the charging device 282. As illustrated, the patient monitor 12 may include the charging device 282. However, in other embodiments, the charging device 282 may also be a device separate from the patient monitor 12. Additionally, while some of the components of the patient monitor 12 and the sensor 14 are not illustrated, it should be noted that the patient monitor 12 and/or the sensor 14 may also include one or more of their respective components as described above with respect to FIG. 2.

The charging device 282 may include a power source 284 configured to provide power to the sensor 14. In certain embodiments, the power source 284 may be configured to provide power for recharging the battery 44 of the sensor 14. The power source 284 may be any suitable power source, such as, for example, a battery, a rechargeable battery, and/or an energy harvesting power supply (e.g., a motion generated energy harvesting device, thermoelectric generated energy harvesting device, or a similar device). Additionally, in some embodiments, the power source 284 may include a power adaptor that is configured to receive power from an external power source 286. The external power source 286 may be an AC power source, a battery, a rechargeable battery, an energy harvesting power supply (e.g., a motion generated energy harvesting device, thermoelectric generated energy harvesting device, or a similar device), and/or any other suitable power source.

The charging device 282 may also include a charging control circuit 288. The charging control circuit 288 may, for example, enable the adaptive control of the power source 284 and/or the power source 286 for providing power to the sensor 14. In certain embodiments, the charging control circuit 288 may also enable or facilitate the determination that the sensor 14 is in proximity to the charging device 282. The charging control circuit 288 may include, for example, a processing circuit and a transceiver 290. In one embodiment, the processing circuit may include the processor 66 of the patient monitor 12. In another embodiment, the processing circuit may be a separate processor from the processor 66. Additionally, in one embodiment, the transceiver 290 may include the transceiver 22 of the patient monitor 12. However, as illustrated, the transceiver 290 may be a separate transceiver from the transceiver 22.

As described above, the sensor 14 may include the one or more additional sensors 48, which may include a proximity detector 292. The proximity detector 292 may include any suitable components for generating information relating to the proximity of the sensor 14 to the charging device 282. By way of example, the proximity detector 292 may include an infrared (IR) sensor, a capacitive photoelectric sensor, an inductive proximity sensor, a Doppler velocimeter, an ultrasonic sensor, or any other suitable sensor. In one embodiment, the proximity detector 292 may include a short range transceiver 294. The transceiver 294 may be configured to at least periodically transmit signals to and/or receive signals from the transceiver 290. Accordingly, in certain embodiments, the transceiver 290 may also be a short range transceiver. The transceiver 290 and the transceiver 294 may be configured to wirelessly transmit data using any suitable wireless standard, such as the IEEE 802.15.4 standard, the Bluetooth standard, one or more of the IEEE 802.11 standards, an ultra-wideband (UWB) standard, or a near-field communication (NFC) standard. In one embodiment, the transceiver 290 and the transceiver 294 may be configured to transmit and/or receive radio-frequency signals.

In some embodiments, the transceiver 294 of the sensor 14 may be configured to transmit signals to the transceiver 290 of the charging device 282, and the charging control circuit 288 (e.g., the processor 66) may be configured to determine that the sensor 14 is in proximity to the charging device 282 if the signals are received by the transceiver 290. In other embodiments, the transceiver 290 of the charging device 282 may transmit signals to the transceiver 294 of the sensor 14, and the processor 112 of the sensor 14 (or another processor or data processing circuitry) may be configured to determine that the sensor 14 is in proximity to the charging device 282 if the signals are received by the transceiver 294. Further, in another embodiment, the transceiver 294 of the sensor 14 may transmit signals to the transceiver 290 of the charging device 282, which, if received by the transceiver 290, may activate the transceiver 290 and cause the transceiver 290 to transmit a confirmation signal to the transceiver 294. In such an embodiment, the processor 112 of the sensor 14 may determine that the sensor 14 is in proximity to the sensor 14 in response to receiving the confirmation signal. Additionally, the transceiver 290 and the transceiver 294 may be configured such that signals transmitted from the transceiver 290 or the transceiver 294 may be received by the transceiver 294 and the transceiver 290, respectively, when the transceiver 290 and the transceiver 294 are within a minimum predetermined distance from the other. For example, the minimum predetermined distance may be between approximately 10 centimeters and 20 meters, 20 centimeters and 15 meters, 30 centimeters and 10 meters, 40 centimeters and 5 meters, 50 centimeters and 4 meters, 60 centimeters and 3 meters, 70 centimeters and 2 meters, or any other suitable range.

In certain scenarios, a weak signal may be received by the transceiver 290 or the transceiver 294 even though the sensor 14 may be located at a distance from the charging device 282 that is greater than the minimum predetermined distance. Accordingly, in some embodiments, it may also be desirable to determine the signal strength of the received signal and to compare the signal strength to a minimum predetermined threshold for signal strength that may indicate that the sensor 14 is in proximity to the charging device 282. The minimum predetermined threshold for signal strength may be empirically determined such that the minimum predetermined threshold for signal strength approximately corresponds to a particular distance between the sensor 14 and the charging device 282. The minimum predetermined threshold for signal strength may be between approximately 0 percent and 100 percent, 20 percent and 95 percent, 40 percent and 90 percent, 60 percent and 85 percent, or any other range. Accordingly, the charging control circuit 288 and/or the processor 112 may be configured to determine the signal strength of the signal received by the transceiver 290 or the transceiver 294, respectively, and may compare the signal strength to the minimum predetermined threshold for signal strength. The charging control circuit 288 and/or the processor 112 may determine that the sensor 14 is in proximity to the charging device 282 if the signal strength is greater than the minimum predetermined threshold for signal strength. Alternatively, if the signal strength is less than the minimum predetermined threshold for signal strength, the charging control circuit 288 and/or the processor 112 may determine that the sensor 14 is not in proximity to the charging device 282.

In another embodiment, the proximity detector 292 may include one or more magnetic field generators 296. In certain embodiments, the magnetic field generator 296 may be configured to produce oscillations (e.g., pulses) such that the magnetic field generator 296 is periodically generating a magnetic field (e.g., magnetic proximity signals). Alternatively, the magnetic field generator 296 may continuously generate a magnetic field. In some embodiments, the magnetic field generator 296 may be configured to generate a magnetic proximity signal having a predetermined signal strength. Accordingly, the charging device 282 may include one or more magnetic field sensors 298 configured to detect magnetic proximity signals. It should be noted, however, that in other embodiments, the sensor 14 may include the magnetic field sensor 298 and the charging device 282 may include the magnetic field generator 296. The charging control circuit 288 may be configured to receive signals from the magnetic field sensor 298 when the magnetic field sensor 298 detects one or more magnetic proximity signals. The charging control circuit 288 may also be configured to determine a signal strength of the received signals and to compare the determined signal strength to a minimum predetermined threshold for signal strength, which may be at least partially dependent upon the predetermined signal strength of the transmitted magnetic proximity signal. The charging control circuit 288 may determine that the sensor 14 is in proximity to the charging device 282 if the signal strength is greater than the minimum predetermined threshold for signal strength. Alternatively, if the signal strength is less than the minimum predetermined threshold for signal strength, the charging control circuit 288 may determine that the sensor 14 is not in proximity to the charging device 282.

Regardless of the method used to determine whether the sensor 14 is in proximity to the charging device 282, the sensor 14 and/or the charging device 282 may be configured to provide a user-perceivable indication of the proximity of the sensor 14 to the charging device 282. The user-perceivable indication may alert the user that the sensor 14 may be coupled to the charging device 282 to receive power from the charging device 282. The user-perceivable indication may be a visual indication and/or an audible indication. For example, the processor 112 of the sensor 14 may cause one of the indicator lights 124 to emit light and/or flash. In some embodiments, the patient monitor 12 and/or the multi-parameter monitor 34 may be configured to provide a visual indication (e.g., a symbol, a graphical indication, and/or a textual message) on the display 30 and/or the display 40, respectively. In one embodiment, the sensor 14 may also include a display (not shown), such as an electronic paper display (e.g., an electronic ink (E-ink) display), which may be configured to display the indication. Additionally, the sensor 14 may include a speaker 300 to provide an audible indication, such as a beep and/or an alarm. The patient monitor 12 may also provide the audible indication via the speaker 74. Further, in some embodiments, the sensor 14 and/or the patient monitor 12 may transmit a signal (e.g., via a central network) to a pager 302 (e.g. a pager of a caregiver), which may cause the pager 302 to provide the user-perceivable indication, which may be visual, audible, and/or tactile (e.g., a vibration).

In some embodiments, it may be desirable to delay the user-perceivable indication for a predetermined period of time, for example, to reduce nuisance alarms on the pager 302. In certain embodiments, the predetermined period of time may be based at least in part upon a level of charge of the battery 44. For example, if the level of charge of the battery 44 is greater than a predetermined charge threshold, the predetermined period of time may be longer than a given predetermined period of time when the level of charge of the battery 44 is less than the predetermined charge threshold. The predetermined charge threshold of the battery 44 may be, for example, between approximately 0 percent and 70 percent, 5 percent and 60 percent, 10 percent and 50 percent, or 15 percent and 40 percent of the total charge level of the battery 44. However, any other suitable charge threshold may be utilized. Similarly, it may be desirable to provide additional user-perceivable indications and/or increase the nuisance or annoyance of the user-perceivable indication if the level of charge of the battery 44 is less than a minimum predetermined charge threshold. For example, the user (e.g., the patient or the caregiver) may not couple the sensor 14 to the charging device 282 in response to a first user-perceivable indication. Thus, it may be desirable to provide a second user-perceivable indication and/or to increase the severity of the first user-perceivable indication (e.g., a louder alarm or flashing indicator light 124) after a predetermined period of time and/or after the level of charge of the battery 44 is less than a minimum predetermined charge threshold.

For example, FIG. 7 illustrates an embodiment of a method 320 for providing indications to couple a sensor (e.g., the sensor 14) to a charging device (e.g., the charging device 282). Certain steps of the method 320 may be performed by a processor, or a processor-based device, such as the sensor 14, the patient monitor 12, and/or the charging device 282, which may each include instructions for implementing certain steps of the method 320. Additionally, the instructions for implementing certain steps of the method 320 may be stored as coded instructions and/or algorithms in a memory device (e.g., the NV memory 108, the RAM 70, and/or the ROM 72) and may be accessed and executed by a processor (e.g., the processor 112, the processor 66, the charging control circuit 288). The method 320 may be implemented with a single user-perceivable indicator or multiple user-perceivable indicators.

The method 320 may include operating a sensor (e.g., the sensor 14) (block 322). The method 320 may also include determining whether the sensor 14 is operating on battery power (e.g., operating in a wireless mode) (block 324). If the sensor 14 is not operating on battery power (e.g., is operating on power received from a charging device or operating in a wired mode), the method 320 may continue sensor operation (block 322). Alternatively, if the processor-based device (e.g., the sensor 14, the patient monitor 12, and/or the charging device 282) determines that the sensor 14 is operating on battery power, the processor-based device may determine whether the sensor 14 is within a predetermined range of a charging device (e.g., the charging device 282) (block 326). Determining whether the sensor 14 is within a predetermined range of the charging device 282 may include utilizing signals from the proximity detector 292, as described above with respect to FIG. 6, to determine whether the sensor 14 is located less than a predetermined distance from the charging device 282. If the sensor 14 is not within the predetermined range of the charging device 282, the method 320 may continue sensor operation (block 322). Alternatively, if the processor-based device (e.g., the sensor 14, the patient monitor 12, and/or the charging device 282) determines that the sensor 14 is within the predetermined range of the charging device 282, the processor-based device may provide a first indication to couple the sensor 14 to the charging device 282 (block 328). As described above, the first indication may be a visual and/or an audible user-perceivable indication. For example, the sensor 14 may cause one or more of the indicator lights 124 to emit light. The sensor 14 may also be configured to emit an audible indication via the speaker 300. In certain embodiments, the first indication to couple the sensor 14 to the charging device 282 (block 328) may be continuously or periodically provided until the sensor 14 is coupled to the charging device 282. Alternatively, the first indication to couple the sensor 14 to the charging device 282 (block 328) may be provided only once.

The method 320 may also include setting a timer (block 330). After the timer has been set (block 330), the method 320 may include determining whether the sensor 14 has been coupled to the charging device 282 (block 332). In certain embodiments, the sensor 14 may determine that the sensor 14 is coupled to the charging device 282 if the sensor 14 detects received power. In response to determining that the sensor 14 is coupled to the charging device 282, the processor-based device (e.g., the sensor 14, the patient monitor 12, and/or the charging device 282) may cancel the timer (block 334) and may continue sensor operation (block 322). If the processor-based device (e.g., the sensor 14, the patient monitor 12, and/or the charging device 282) determines that the sensor 14 is not coupled to the charging device 282, the timer may continue to run. The method 320 may include determining whether the timer has expired (block 336). If the timer has not expired, the method 320 may continue determining whether the sensor 14 is coupled to the charging device 282 (block 332).

However, if the timer expires, the processor-based device (e.g., the sensor 14, the patient monitor 12, and/or the charging device 282) may provide a second indication to couple the sensor 14 to the charging device 282 (block 338). In certain embodiments, the second indication (block 338) may be different than the first indication (block 328). In particular, providing the second indication (block 338) may include escalating a property of the first indication (block 328). For example, where the first indication (block 328) was provided as an audible tone, the second indication (block 338) may be provided as an audible tone with a higher frequency and/or a higher volume. In another embodiment, the harmonic content of the audible tone may change for the second indication (block 338). Additionally or alternatively, where the first indication (block 328) included emitting one of the indicator lights 124, the second indication (block 338) may include flashing the indicator light 124 and/or emitting another of the indicator lights 124. In some embodiments, the first indication (block 328) may be provided by the sensor 14, and the second indication (block 338) may be provided by the patient monitor 12, the multi-parameter monitor 34, the charging device 282, and/or the pager 302. Additionally, if the timer has expired, the timer may be reset with a reduced period (e.g., half of the initial timer period) (block 340). In some embodiments, this procedure continues as each successive timer expires, thus gradually increasing the annoyance level of the second indication (block 338), which may be desirable in some embodiments to alert a user to couple the sensor 14 to the charging device 282.

The disclosed embodiments may be interfaced to and controlled by a computer readable storage medium having stored thereon a computer program. The computer readable storage medium may include a plurality of components such as one or more of electronic components, hardware components, and/or computer software components. These components may include one or more computer readable storage media that generally store instructions such as software, firmware and/or assembly language for performing one or more portions of one or more implementations or embodiments of an algorithm as discussed herein. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or solid-state or electronic memory. Other forms of non-transitory and/or tangible computer readable storage media not list may be employed with the disclosed embodiments.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims

What is claimed is:

1. A sensor configured to operate in a wireless mode or in a wired mode, comprising:
one or more sensing components configured to measure a physiological signal of a patient;
a transceiver configured to wirelessly transmit the physiological signal at least when the sensor is operating in the wireless mode;
a power source configured to power the one or more sensing components and the transceiver at least when the sensor is operating in the wireless mode;
a connector configured to receive a cable, wherein the cable is configured to couple to a charging device configured to provide power to the one or more sensing components of the sensor or to recharge the power source of the sensor, and wherein the sensor is configured to operate in the wired mode when the connector is coupled to the cable; and
a proximity detector configured to generate one or more signals relating to a distance between the sensor and the charging device.

2. The sensor of claim 1, wherein the sensor comprises processing circuitry configured to receive the one or more signals from the proximity detector and to determine whether the sensor is located within a predetermined distance of the charging device based at least in part upon the one or more signals.

3. The sensor of claim 2, wherein the processing circuitry is configured to provide a user-perceivable indication via a first indicator in response to determining that the sensor is located within the predetermined distance of the charging device and that the sensor is operating in the wireless mode.

4. The sensor of claim 3, wherein the sensor comprises one or more indicator lights, and wherein the processing circuitry is configured to cause one of the one or more indicator lights to emit light to provide the user-perceivable indication.

5. The sensor of claim 3, wherein the processing circuitry is configured to determine whether the sensor is coupled to the charging device within a predetermined period of time after the user-perceivable indication is provided, and wherein the processing circuitry is configured to provide a second user-perceivable indication via the first indicator or a via a second indicator in response to determining that the sensor was not coupled to the charging device within the predetermined period of time.

6. The sensor of claim 1, wherein the proximity detector comprises a short range transceiver configured to wirelessly communicate with a short range transceiver of the charging device.

7. The sensor of claim 1, wherein the sensor comprises a pulse oximetry sensor.

8. A method for providing indications to charge a medical sensor, comprising:
determining whether the medical sensor is operating in a wireless mode;
determining whether the medical sensor is within a predetermined distance of a charging device that is configured to provide power to the medical sensor if the medical sensor is operating in the wireless mode; and
providing a first indication to couple the medical sensor to the charging device in response to determining that the medical sensor is operating in the wireless mode and that the medical sensor is within the predetermine distance of the charging device.

9. The method of claim 8, wherein providing the first indication comprises emitting light from at least one indicator light disposed on the medical sensor.

10. The method of claim 8, wherein providing the first indication comprises providing an audible alarm from a speaker of the medical sensor or a speaker of the charging device.

11. The method of claim 8, wherein providing the first indication comprises providing a visual indication on a display of a patient monitoring device operatively coupled to the medical sensor.

12. The method of claim 8, comprising:
determining whether the medical sensor was coupled to the charging device within a predetermined period of time; and
providing a second indication to couple the medical sensor to the charging device in response to determining that the medical sensor was not coupled to the charging device within the predetermined period of time.

13. The method of claim 12, wherein the second indication is provided by a pager in remote communication with the medical sensor or the charging device.

14. The method of claim 12, wherein the first indication is provided as an audible tone with a first property and the second indication is provided as an audible tone with a second property.

15. The method of claim 12, wherein the first indication is provided as an audible tone with a first volume and the second indication is provided as an audible tone with a second volume that is greater than the first volume.

16. A system, comprising:
a charging device; and
a sensor configured to measure a physiological signal of a patient, wherein the sensor is configured to operate in a wireless mode and a wired mode, and wherein the sensor comprises:
a power source configured to power the sensor when the sensor is operating in the wireless mode;
a proximity detector configured to generate one or more signals relating to a distance between the sensor and the charging device; and
a transceiver configured to wirelessly transmit the physiological signal or the one or more signals generated by the proximity detector at least when the sensor is operating in the wireless mode; and
wherein the charging device is configured to provide power to the sensor when the sensor is coupled to the charging device.

17. The system of claim 16, comprising a monitor configured to receive the physiological signal from the sensor, wherein the monitor comprises a processor configured to calculate a physiological parameter of the patient based at least in part upon the signal, and wherein the monitor comprises the charging device.

18. The system of claim 17, wherein the sensor comprises a pulse oximetry sensor, and wherein the physiological parameter comprises blood oxygen saturation.

19. The system of claim 17, wherein the monitor comprises a transceiver configured to receive, from the transceiver of the sensor, the one or more signals generated by the proximity detector, and wherein the processor is configured to determine whether the sensor is located within a predetermined distance of the monitor based at least in part upon the one or more signals.

20. The system of claim 19, wherein the monitor comprises a display, and wherein the processor is configured to cause the display to display an indication to couple the sensor to the monitor.
